(19) United States
(12) Patent Application Publication
(10) Pub. No.: US 2008/0305498 A1
(43) Pub. Date: Dec. 11, 2008

(54) Biomarkers for Use in Vessel Disorders

(75) Inventors: Dianna M. Milewicz, Houston, TX (US); William P. Dubinsky, Houston, TX (US)

Correspondence Address: FULBRIGHT & JAWORSKI L.L.P, 2200 ROSS AVENUE, SUITE 2800, DALLAS, TX, 75201-2784, US

(73) Assignee: BOARD OF REGENTS, THE UNIVERSITY OF TEXAS, AUSTIN, TX (US)

(21) Appl. No.: 11/577,287

(22) PCT Filed: Oct. 14, 2005
(86) PCT No.: PCT/US2005/036789

§ 371 Date: Aug. 1, 2008

Related U.S. Application Data
(60) Provisional application No. 60/618,721, filed on Oct. 14, 2004.

(51) Int. Cl. G01N 33/554; C12Q 1/02

(52) U.S. Cl. 435/7.21; 435/29

(57) ABSTRACT

The present invention relate to a method of diagnosing a subject having a vessel disorder. More particularly, a biological sample is obtained from a subject suspected of having a vessel disorder. A protein profile is determined or measured in the sample using procedures described herein, for example, mass spectrometry or immunodetection. The protein profile from the subject is compared to a protein profile in a healthy control, wherein an alteration in the levels of a protein of the profile in the subject compared to the healthy control is indicative of a vessel disorder.

### CROSS-REFERENCE TO RELATED APPLICATIONS

This application claims priority to U.S. Provisional Application No. 60/618,721 filed Oct. 14, 2004, which is incorporated herein by reference in its entirety.

### TECHNICAL FIELD

The present invention relates to the use of a biomarker or a specific protein profile to detect or diagnose a subject suffering from or suspected of having a vessel disorder.

### BACKGROUND OF THE INVENTION

Proper functioning of the vascular system is essential for the health and fitness of living organisms. The vascular system carries essential nutrients and blood gases to all living tissues and removes waste products for excretion. The vasculature is divided into different regions depending on the organ systems served. If vessels feeding a specific organ or group of organs are compromised, the organs and tissues supplied by those vessels are deleteriously affected and may even fail completely.

Disorders of the peripheral arterial system cause their pathological effects by two general mechanisms: obstruction of the arterial lumen or disruption of the vessel wall. Arterial obstruction most commonly results from atherosclerosis, although other causes of luminal blockage may be identified, including inflammatory conditions, external compression, emboli, thrombi or fibromuscular dysplasia. Arterial obstructive disorders typically affect multiple sites within the arterial tree. Disruption of the arterial wall results in aneurysm formation, arterial wall dissection or frank arterial rupture. Arterial aneurysm formation is most commonly related to atherosclerosis but is also associated with a poorly understood pathology termed cystic medial necrosis. Aneurysms may also result from infections, congenital anomalies or trauma. Aneurysms predispose the aorta to dissection or rupture. Arterial wall dissection may arise as a complication of an aneurysm or as an independent event. Arterial wall dissection in the absence of a pre-existing aneurysm may occur spontaneously, or it may result from trauma. Aneurysms can be asymptomatic until an event like dissection or rupture occurs. Frank arterial rupture may result from the progressive expansion of an arterial aneurysm beyond a certain critical diameter. If no aneurysm is present, the most common cause of arterial rupture is some type of arterial trauma. Artery wall disruption may affect multiple sites, or may be localized to only one location.

Dissecting aortic aneurysm is a disease with severe chest pains and is caused by disruption of the aortic media by blood entering through a laceration of the luminal vascular wall. Dissections can also occur without a laceration of the luminal vessel wall. In these cases, an intramural hemotoma is present without evidence of a laceration of the luminal wall. A dissecting aortic aneurysm occurs in the aorta in most cases, but it also can occur in branches in some cases. As to the causes for the disease, it is caused not only the degeneration and weakening of the media (e.g., cystic medionecrosis and arterio-sclerosis) but also the extension of aorta, hypertension, etc.

A typical example of disease with chest pains is acute myocardial infarction. Acute myocardial infarction may be diagnosed without much difficulty through electrocardiograph change or biochemical blood testing. By contrast, in the case of dissecting aortic aneurysm, a specific change is hardly observed in an electrocardiograph or blood testing in spite of the high lethality of this disease. Therefore, diagnosis of this disease requires extreme care.

As a method for diagnosis of dissecting aortic aneurysm, echo examination, CT (X-rays computed tomography), DSA (digital subtraction angiography), MRI (magnetic resonance imaging), etc. have been attempted and have produced rather good results (Nankodo K. K.). All of these methods, however, require special equipment, and hence are not always satisfactory as a method for use in an urgent examination wherein the method is required to be carried out anywhere.

Accordingly, the present invention is mainly intended to provide a method for detecting vessel disorders, such as dissecting aortic aneurysm, which are applicable to an urgent examination, and a reagent for use in the method

### BRIEF SUMMARY OF THE INVENTION

Certain embodiments of the present invention relate to a method of diagnosing a subject having a vessel disorder. In diagnosing a subject suspected of having a vessel disorder, a biological sample, for example a blood sample, saliva sample, or tissue sample, is obtained from the subject. A protein profile is determined or measured in the sample using procedures described herein, for example, mass spectrometry, immunodetection (e.g., antibody binding for example, ELISA or Western blotting), etc. The protein profile can comprise at least one protein or more than one protein. The proteins in the protein profile can include, for example, but are not limited to alpha-1 antitrypsin, transthyretin, apolipoprotein-a-1, serum amyloid A, haptoglobin, haptoglobin precursor, haptoglobin fragments, fibrinogen A, fibrinogen B, fibrinogen G, annexin AI, complement C4, complement factor B, apolipoprotein A1, apoliprotein B-100, macroglobulin A2, proteo cadherin gamma A6, transferrin, transferrin fragments, and a combination thereof. Yet further, proteins in the protein profile may also be identified purely by their atomic mass, for example, the proteins can have an atomic mass of about 2,500 daltons to about 120,000 daltons. The protein profile from the subject is compared to a protein profile in a healthy control, wherein an alteration in the levels of a protein of the profile in the subject compared to the healthy control is indicative of a vessel disorder. One of skill in the art understands that the present invention comprises a method of determining the amounts or level of a protein profile, as such the amounts or levels can be determined quantitatively or qualitatively. Thus, the present invention encompasses determining merely the presence and/or absence of a protein or protein profile, as well as the quantitative amounts of a protein profile in a suspected subject versus a health subject.

An embodiment of the present invention comprises a protein profile that is indicative of a vessel disorder in a subject and comprises altered amounts of one or more proteins selected from the group consisting of alpha-1 antitrypsin, transthyretin, apolipoprotein-a-1, serum amyloid A, haptoglobin, haptoglobin precursor, haptoglobin fragments, fibrinogen A, fibrinogen B, fibrinogen G, annexin AI, complement C4, complement factor B, apolipoprotein A1, apoliprotein B-100, macroglobulin A2, proteo cadherin gamma A6, transferrin, transferrin fragments, and a combination thereof.

Yet further, in certain embodiments of the present invention, the protein profile contains a protein or biomarker that is indicative of a vessel disorder. For example the presence and/or absence of a protein or biomarker indicates a vessel disorder. Proteins that can be used as a biomarker include, for example, alpha-1 antitrypsin, transthyretin, apolipoprotein-a-1, serum amyloid A, haptoglobin, haptoglobin precursor, haptoglobin fragments, fibrinogen A, fibrinogen B, fibrinogen G, annexin AI, complement C4, complement factor B, apolipoprotein A1, apoliprotein B-100, macroglobulin A2, proteo cadherin gamma A6, transferrin, transferrin fragments, and a combination thereof. More particularly, the protein or biomarker is haptoglobin, haptoglobin precursor, haptoglobin fragments, or any combination thereof.

Another embodiment of the present invention comprises a protein profile that is indicative of a vessel disorder in a subject and comprises altered amounts of one or more proteins having an atomic mass in daltons selected from the group consisting of 1,171, 1,177, 1,384, 2,796, 5,738, 5,761, 6,380, 8,707, 9,406, 10,388, 10,964, 10,977, 11,364, 11,410, 11,446, 11,497, 11,669, 11,743, 13,589, 16,000, 16,001, 19,459, 20,000, 30,778, 31,358, 45,177, 45,294, 45,595, 46,091, 46,160, 46,707, 60,279, 67,174, 67,307, 92,112, 114,287 or any range therebetween.

In certain embodiments, the protein profile may comprise altered amounts of one or more proteins having an atomic mass in daltons in the range of about 2,500 daltons to about 6,500 daltons; 6,000 daltons to about 12,000 daltons; 25,000 daltons to about 45,000 daltons; 45,000 daltons to about 65,000 daltons; 65,000 daltons to about 95,000 daltons; about 95,000 daltons to about 120,000 or any range therebetween. More specifically, the proteins may comprise an atomic mass of 2,796.0 daltons, 5,763.0 daltons, 5,816.8 daltons, 5837.7 daltons, 6,380.3 daltons, 8,097.0 daltons, 10,390.5 daltons, 10,983.5 daltons, 11,074.5 daltons, 11,423.1 daltons, 11,649.1 daltons, 11,828.1 daltons, 10,983.5 daltons, 11,649.1 daltons, 28,391.3 daltons, 29,212.7 daltons, 31,358.4 daltons, 44,792.5 daltons, 45,313.3 daltons, 45,595.8 daltons, 45,937.1 daltons, 46,005.0 daltons, 46,160.6 daltons, 46,182.4 daltons, 66,549.9 daltons, 67,251.0 daltons, 37,320.3 daltons, 92,112.6 daltons, 100,901.8 daltons, and 114,287.1 daltons.

The vessel disorder is selected from the group consisting of dissection, aneurysm, inflammatory aortic disease, pseudoaneurysm, and false aneurysm. More specifically, the dissection is Type A or Type B, with the dissection being initiated in the ascending or descending aorta, respectively.

Another embodiment of the present invention comprises a protein profile that is indicative of a Type A aortic dissection in a subject and comprises altered amounts of one or more proteins having an atomic mass in daltons selected from the group consisting of 2,796.0, 5,763.0, 5,816.8, 5,837.7, 6,380.3, 8,097.0, 10,983.5, 11,074.5, 11,423.1, 11,649.1, 11,828.1, 28,391.3, 29,212.7, 3,1358.4, 44,792.5, 45,313.3, 45,595.8, 46,160.6, 67,251.0, 67,320.3, 100,901.8, 114,287.1, and any range therebetween.

Still further, another embodiment of the present invention comprises a protein profile that is indicative of a Type A aortic dissection in a subject and comprises altered amounts of one or more proteins selected from the group consisting of alpha-1 antitrypsin (about 43 kDa), serum amyloid A, haptoglobin precursor, haptoglobin fragment, fibrinogen A, fibrinogen B, fibrinogen G, annexin AI, complement C4, transferrin and a combination thereof.

Yet further, another embodiment of the present invention comprises a protein profile that is indicative of a Type B aortic dissection in a subject and comprises altered amounts of one or more proteins having an atomic mass in daltons selected from the group consisting of 10,390.5, 10,983.5, 11,649.1, 11,828.1, 31,358.4, 44,792.5, 45,313.3, 45,937.1, 46,005.0, 46,160.6, 46,182.4, 66,549.9, 67,251.0, 67,320.3, 92,112.6, 114,287.1, and any range therebetween.

Another embodiment of the present invention comprises a protein profile that is indicative of a Type B aortic dissection in a subject and comprises altered amounts of one or more proteins selected from the group consisting of serum amyloid A, haptoglobin B, and a combination thereof.

Still further, another embodiment of the present invention comprises a method of diagnosing a subject having a vessel disorder comprising the steps of: obtaining a sample from the subject; determining a protein profile in the sample; and comparing the protein profile of the same proteins in a healthy control, wherein an alteration in the levels of a protein of the profile in the subject compared to the healthy control is indicative of a vessel disorder. The sample can be a blood sample, a saliva sample, a plasma sample or a tissue sample. Still further, the determining step can comprise such methods as mass spectrometry and/or immunodetection e.g., antibody binding, for example ELISAs or Western blotting. The alteration can include either the presence and/or absence of a protein or an increase and/or decrease in the amount or level of a protein in the healthy control compared to the sample from the subject.

In certain embodiments, the determination step can be based upon a statistical basis followed by a visual inspection of the data. The statistical measure is a p value <0.05 as significant differences between the two populations, the healthy control and the sample from the subject. Alternatively, the protein profile is measured qualitatively, for example, alteration in the levels of a protein in the profile refers to the presence or absence of a protein. Thus, the diagnosis can also be based upon qualitative measurements. In certain embodiments, determination of the protein profile is more quantitative for example, the analysis is determined upon a fold difference or fold increase in the amount of at least one protein in the sample from the subject compared to the healthy control sample. The fold difference can be for example, at least a 2-fold increase, at least a 3-fold increase, at least a 4-fold increase, at least a 5-fold increase, at least a 6-fold increase, at least a 7-fold increase, at least a 8-fold increase, at least a 9-fold increase, at least a 10-fold increase, at least a 11-fold increase, at least a 12-fold increase, at least a 13-fold increase, at least a 14-fold increase, at least a 15-fold increase, at least a 20-fold increase, at least a 25-fold increase, at least a 30-fold increase, at least a 35-fold increase, at least a 40-fold increase, at least a 45-fold increase, at least a 50-fold increase, at least a 60-fold increase, at least a 70-fold increase, at least a 80-fold increase, at least a 90-fold increase, at least a 100-fold increase or any range therebetween.

Another embodiment comprises a method of monitoring a subject having or suspected of having a vessel disorder comprising the steps of: obtaining a first sample from the subject at a first selected time point; determining a protein profile in the first sample; obtaining a second sample from the subject at a second selected time point; determining a protein profile in the second sample; comparing the protein profile obtained from first sample and the second sample to assess the change in the profile between the first selected time point and the second selected time point.

A further embodiment comprises a method of diagnosing a subject suffering from a dissection comprising the steps of: obtaining a sample from the subject; determining in the sample a protein profile, wherein the protein profile comprises one or more proteins having an atomic mass in daltons in the range of about 2,500 daltons to about 6,500 daltons; 6,000 daltons to about 12,000 daltons; 25,000 daltons to about 45,000 daltons; 45,000 daltons to about 65,000 daltons; 65,000 daltons to about 95,000 daltons; about 95,000 daltons to about 120,000 or any range therebetween. More specifically, the proteins may comprise an atomic mass of 2,796.0, 5,763.0, 5,816.8, 5837.7, 6,380.3, 8,097.0, 10,390.5, 10,983.5, 11,074.5, 11,423.1, 11,649.1, 11,828.1, 10,983.5, 11,649.1, 28,391.3, 29,212.7, 31,358.4, 44,792.5, 45,313.3, 45,595.8, 45,937.1, 46,005.0, 46,160.6, 46,182.4, 66,549.9, 67,251.0, 37,320.3, 92,112.6, 100,901.8, 114,287.1, and a combination thereof; and comparing the protein profile of the same proteins in a healthy control, wherein an alteration in the levels of a protein of the profile in the subject compared to the healthy control is indicative of a dissection.

Another embodiment of the present invention comprises a method of diagnosing a subject suffering from an Type A aortic dissection comprising the steps of: obtaining a blood sample from the subject; determining in the sample a protein profile, wherein the protein profile comprises one or more proteins having an atomic mass in daltons selected from the group consisting of 2,796.0, 5,763.0, 5,816.8, 5,837.7, 6,380.3, 8,097.0, 10,983.5, 11,074.5, 11,423.1, 11,649.1, 11,828.1, 28,391.3, 29,212.7, 3,1358.4, 44,792.5, 45,313.3, 45,595.8, 46,160.6, 67,251.0, 67,320.3, 100,901.8, 114,287.1 and any range therebetween; and comparing the protein profile of the same proteins in a healthy control, wherein an alteration in the levels of one or more proteins of the profile in the subject compared to the healthy control is indicative of a Type A aortic dissection.

Still further, another embodiment comprises a method of diagnosing a subject suffering from an Type B aortic dissection comprising the steps of: obtaining a blood sample from the subject; determining in the sample a protein profile, wherein the protein profile comprises one or more proteins having an atomic mass in daltons selected from the group consisting of 10,390.5, 10,983.5, 11,649.1, 11,828.1, 31,358.4, 44,792.5, 45,313.3, 45,937.1, 46,005.0, 46,160.6, 46,182.4, 66,549.9, 67,251.0, 67,320.3, 92,112.6, 114,287.1, and any range therebetween; and comparing the protein profile of the same proteins in a healthy control, wherein an alteration in the levels of a protein of the profile in the subject compared to the healthy control is indicative of a Type B aortic dissection.

In further embodiments of the present invention, the protein profile is packages as a kit for detecting a vessel disorder comprising a container having the protein profile of the present invention.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 is a typical spectrum obtained on a cation exchange chip of plasma proteins that bind at pH 4.0.

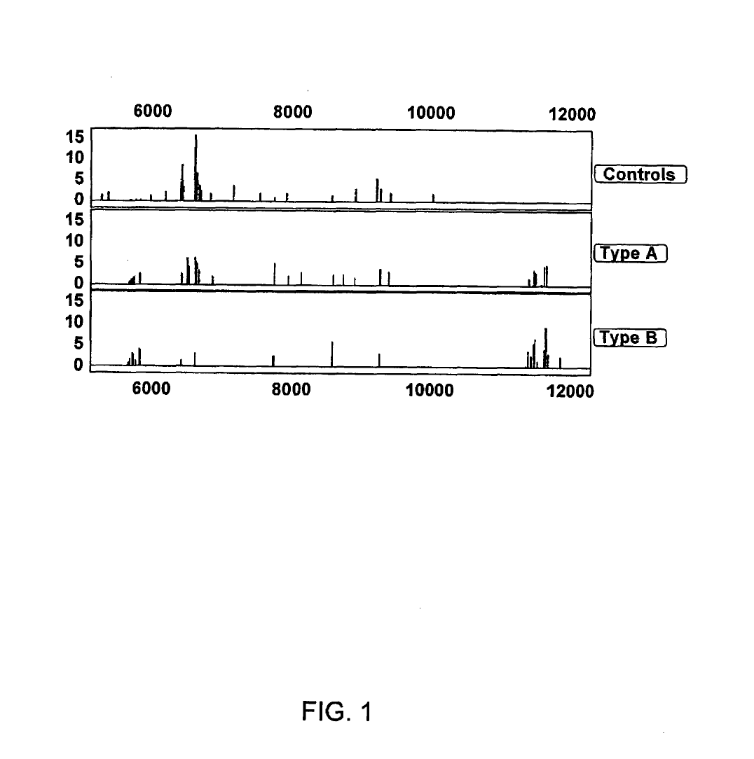

FIG. 2 shows identification of elevated Haptoglobin and a fragment in Type A dissection patient plasma. Fraction V from Hyper Q DF anion exchange resin in 96 well plate eluted with 50 mM sodium citrate, 0.1% octylglucopyranoside, pH 3.0.

FIG. 3 shows identification of Haptoglobin fragments of different masses in plasma from Type A dissection patients compared to control. The approximate molecular weights are 19 kDa in the Type A dissection and 15 kDa in the control. Fraction VI from Hyper Q DF anion exchange resin in 96 well plate eluted with isopropanol:acetonitrile:TFA (33%:16.7%:0.1% in water).

FIG. 4 shows identification of haptoglobin fragments and serum amyloid in plasma from Type B Dissection. There is a mass difference in the Haptoglobin fragments between the control and Type A dissection. The Type A dissection is approximately 19 kDa compared to 15 kDa in the control. Type Fraction VI from Hyper Q DF anion exchange resin in 96 well plate eluted with isopropanol:acetonitrile:TFA (33%:16.7%:0.1% in water).

FIG. 5 shows elevated levels of Fibrinogen G in plasma from Type A dissection patients. Haptoglobin fragment (19 kDa) also present in this fraction. Fraction VI from Hyper Q DF anion exchange resin in 96 well plate eluted with isopropanol:acetonitrile:TFA (33%:16.7%:0.1% in water).

FIG. 6 shows elevated levels of Fibrinogen B and Fibrinogen G in plasma from Type A dissection patients. Fraction VI from Hyper Q DF anion exchange resin in 96 well plate eluted with isopropanol:acetonitrile:TFA (33%:16.7%:0.1% in water).

FIG. 7 shows identification of elevated Fibrinogen A in Type A dissection patient plasma. Fraction V from Hyper Q DF anion exchange resin in 96 well plate eluted with 50 mM sodium citrate, 0.1% octylglucopyranoside, pH 3.0.

FIG. 8 shows identification of Transferrin fragment in Type A dissection patient plasma. Fraction I from Hyper Q DF anion exchange resin in 96 well plate eluted with 50 mM Tris-HCl, 0.1% octylglucopyranoside, pH 9.0.

FIG. 9 shows identification of a-1-antitrypsin fragment in Type A dissection patient plasma. There is a decrease in the intact a-1-antitrypsin compared to control and an increase in a readily identified band in the Type A. There is also a 19 kDa haptoglobin fragment in the Type A. Fraction V from Hyper Q DF anion exchange resin in 96 well plate eluted with 50 mM sodium citrate, 0.1% octylglucopyranoside, pH 3.0.

FIG. 10 shows identification of increased Complement C4 and Annexin A1 in plasma from Type A dissection patients compared to control. α-1-antitrypsin was also localized to the Annexin A1 band. Fraction IV from Hyper Q DF anion exchange resin in 96 well plate eluted with 100 mM sodium acetate, 0.1% octylglucopyranoside, pH 4.0.

### DETAILED DESCRIPTION OF THE INVENTION

### I. Definitions

Unless defined otherwise, technical and scientific terms used herein have the same meaning as commonly understood by one of ordinary skill in the art to which this invention belongs. For purposes of the present invention, the following terms are defined below.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having”, “including”, “containing” and “comprising” are interchangeable and one of skill in the art is cognizant that these terms are open ended terms.

As used herein, the term “arterial wall disruptive disorder” refers to those abnormalities of arterial walls characterized by the formation of aneurysms or by the formation of disruptions such as dissections. Arterial wall disruptive disorders included disorders associated with the aorta or arterials. Examples of aortic disorders include, but are not limited to aortic aneurysm (e.g., true aneurysm, pseudoaneurysm, or false aneurysm), aortic dissection (e.g., Type A or Type B), aortic occlusion, aortitis, or rupture of the aorta. Examples of arterial disorders include, but are not limited to occlusion (e.g., atherosclerosis, thromboangiitis obliterans, acute arterial occlusion, atheroembolism), aneurysms, dissection, rupture, thoracici outlet compression syndrome, ateriovenous fistulas, Raynaoud's phenomenon, acrocyanosis, livedo reticularis, pemio, and erthyromelalgia.

As used herein, the term “artery” or “arterial” refers to a blood vessel conveying blood away from the heart. These vessels are relatively thick-walled and muscular.

As used herein, the term “vein” or “venous” refers to a blood vessel conveying blood toward the heart.

As used herein, the term “vessel disorder” refers to those abnormalities of any vessel, such as arterials or venous vessels or lymphatics characterized in a disruption of any layer of the vessel wall, such as the tunica intima (endothelium), tunica media (smooth muscle and elastic tissue) or tunica adventitia (fibrous connective tissue). Exemplary vessel disorder includes arterial wall disruptive disorders (e.g., dissections (Type A and/or Type B), aneurysms, occlusions, etc.) and venous disorders (e.g., venous thrombosis).

As used herein, the term “vessel” refers to a structure conveying or containing a liquid, such as a blood. Vessels can include blood vessels and/or lymph vessels.

As used herein, the term “blood vessel” refers to any vessel conveying blood, for example, arteries, arterioles, capillaries, venules, and veins.

As used herein, the term “marker” or “biomarker” refers to a molecule or molecules, such as a protein or polypeptide or a small molecule, that is present in a sample obtained from a subject suffering from a disease of interest in an amount that differs from the amount present in a sample from a “normal”, non-diseased subject. Proteins, polypeptides, or small molecules used as markers in the present invention are contemplated to include any fragments thereof.

As used herein, the term “protein profile,” “protein panel” or “panel” refers to at least one, but preferably two or more molecules, such as proteins, that are markers or biomarkers for a particular disease. The “panel” for a particular disease is preferably selected such that a particular “profile” of marker levels is specific for that disease and capable of clearly distinguishing disease from non-disease. The panel may include any practical number of markers appropriate for use with the diagnosis of the particular one or more diseases or conditions.

As used herein, the term “sample” refers to a biological sample, more particular a blood sample, serum or plasma sample or other bodily fluids (e.g., saliva, etc.), a tissue biopsy and/or aspiration of tissue or cells during a surgical procedure. In certain embodiments, the biological sample is a tissue sample, which may comprise a cell, for example, a vessel cell, for example, a cell from the aorta. One of skill in the art realizes that a plasma sample contains the proteinaceous fluid or non-cellular portion of the blood sample.

As used herein, the term “diagnosis” refers to methods by which the skilled artisan can estimate and/or determine whether or not a subject is suffering from a given disease or condition. The skilled artisan often makes a diagnosis on the basis of one or more diagnostic markers, the presence, absence, or amount of which may be indicative of the presence, severity, or absence of the condition. In addition to markers, other tests, such as ECG, Echo, and MRI, and other factors, such as patient's history, sex, age, and race, may also be used in making the diagnosis.

As used herein, the term “healthy control” or “negative control” or “non-diseased” refers to a sample obtained from a subject known not to be suffering from a vessel disorder or a genetically-induced vessel disorder a sample obtained previously from the subject prior to the onset or suspicion of the vessel disorder or a genetically-induced vessel disorder or a standard from data obtained from a data bank corresponding to currently accepted normal states of these proteins.

As used herein, the term “diseased control” or “positive control” refers to a sample obtained from a subject known to be suffering from a vessel disorder or a genetically-induced vessel disorder. Proteins of this profile for a vessel disorder and genetically-induced vessel disorder were identified using a sub-fractionation procedure and focused proteomic analysis.

As used herein, the term “trauma” includes both non-iatrogenic and iatrogenic processes.

### II. Development of a Profile

In practice, data may be obtained from a group of subjects. The subjects may be patients who have been tested for the presence or level or amount of certain markers. Thus, the markers are measured or determined quantitatively or qualitatively. A particular set of markers may be relevant to a particular condition or disease. The method is not dependent on the actual markers. The markers discussed in this document are included only for illustration and are not intended to limit the scope of the invention.

The group of subjects is divided into at least two sets. The first set includes subjects who have been confirmed as having a disease. For example, this first set of subjects may be those that have had an aneurysm, vessel rupture or dissection. Hereinafter, subjects in this first set will be referred to as “diseased”.

The second set of subjects are selected from those who do not fall within the first set. This set may include all remaining subjects, or only those subjects being in a second condition state. Subjects in this second set will hereinafter be referred to as “non-diseased” or “healthy”. The first and second sets of data are said to be a group of data. Multiple groups of data may be defined by repeating the steps above for different disease states, condition states, or any other selection criteria.

The data obtained from subjects in these sets includes levels of a plurality of markers. Preferably, data for the same set of markers is available for each patient. This set of markers may include all candidate markers, which may be suspected as being relevant to the detection of a particular disease or condition. Actual known relevance is not required. Embodiments of the methods and systems described herein may be used to determine which of the candidate markers are most relevant to the diagnosis of the disease or condition.

The marker, more preferably, is a proteineous compound, for example, a protein or polypeptide, or peptide fragment determined from a biological sample, for example a blood sample (e.g., serum or plasma), other bodily fluids (e.g., saliva, etc.), a tissue biopsy and/or aspiration of tissue or cells during a surgical procedure. In certain embodiments, the biological sample is a tissue sample, which may comprise a cell, for example, a vessel cell, for example, a cell from the aorta. Biological samples are obtained from the subjects in both sets.

Proteins in the sample are then fractionated based on at least one physio-chemical property of the protein. For example, the identity of the protein will indicate several predicted physio-chemical characteristics of the protein. Amino acid sequence will provide a predicted molecular mass of the protein. The amino acid sequence also can be used to predict the isoelectric point of the protein, whether the protein is hydrophilic or hydrophobic and whether the protein has metal chelate binding ability. Amino acid sequence also can indicate whether the protein includes glycosylation or phosphorylation sites. Post-translational modifications of the protein will be reflected in changes to molecular weight. Amino acid sequence also can identify epitopes which, in turn, may be targets for antibody binding. An exact measurement of the physiochemical property is not necessary; it is sufficient to obtain some information so that upon fractionation into a plurality of aliquots based on that characteristic, the protein is expected to be preferentially fractionated among the aliquots.

The proteins in the sample are then fractionated based on a physiochemical characteristic of the protein. A most useful method of separation is molecular weight, as there are many useful methods to separate proteins based on this characteristic including, for example, SDS gel electrophoresis, gas phase ion spectrometry. Another useful physiochemical characteristic is isoelectric point. Isoelectric focusing, affinity chromatography and solid phase extraction on an ion exchange resin will fractionate proteins in a sample based on this property.

Methods of fractionating proteins are used to examine the level of expression of a selected protein in a cell. As described above, the use of one or more elected physiochemical characteristics can enhance the sensitivity of fractionation and reduce background. The techniques described herein can be used to examine one or more proteins expressed in a cell, up to tens, hundreds, thousands, or tens of thousands of proteins. Any one technique or a combination of techniques can be used to fractionate the proteins, based on one or more physio-chemical property. Methods of fractionation include, e.g., two dimensional gels; capillary gel electrophoresis; mass spectrometry, e.g., MALDI, SELDI; ICAT (isotope coded affinity tag, see, e.g., Mann, 1999; Gygi et al., 1999); chromatography, e.g., gel-filtration, ion-exchange, affinity, immunoaffinity, and metal chelate chromatography, HPLC, e.g., reversed phase, ion-exchange, and size exclusion HPLC (see, e.g., Blackstock & Weir, 1999; Dutt & Lee, April 2000; Page et al., 1999; Wang & Hewick, 1999; Regnier et al., 1999; and Pandey & Mann, 2000). The proteins of interest are identified and isolated using techniques known to those of skill in the art.

A. Mass Spectrometry Analysis of Samples

Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). For low molecular weight molecules, mass spectrometry has been part of the routine physical-organic repertoire for analysis and characterization of organic molecules by the determination of the mass of the parent molecular ion. In addition, by arranging collisions of this parent molecular ion with other particles (e.g., argon atoms), the molecular ion is fragmented forming secondary ions by the so-called collision induced dissociation (CID). The fragmentation pattern/pathway very often allows the derivation of detailed structural information. Other applications of mass spectrometric methods known in the art are summarized in Methods in Enzymology, Vol. 193: “Mass Spectrometry” (J. A. McCloskey, editor), 1990, Academic Press, New York.

The proteins of the invention or fragments thereof can be analyzed using mass spectrometry methods. This method fractionates the proteins based on mass. In certain embodiments gas phase ion spectrophotometer is used. In other embodiments, laser-desorption/ionization mass spectrometry is used to analyze the sample on the substrate-bound adsorbent.

Modern laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). Mass spectrometers utilizing laser desorption/ionization mass spectrometry can be further coupled to a quadrupole time-of-flight mass spectrometer. In MALDI, the analyte, which may contain biological molecules, is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait), each of which are incorporated herein by reference in its entirety.

In SELDI, the substrate surface is modified so that it is an active participant in the desorption process. In one variant, the surface is derivatized with affinity reagents that selectively bind the analyte. In another variant, the surface is derivatized with energy absorbing molecules that are not desorbed when struck with the laser. In another variant, the surface is derivatized with molecules that bind the analyte and that contain a photolytic bond that is broken upon application of the laser. In each of these methods, the derivatizing agent generally is localized to a specific location on the substrate surface where the sample is applied. See, e.g., U.S. Pat. Nos. 5,894,063, 6,734,022, 6,528,320, 6,124,137, 6,027,942, 5,719,060 and 5,6020208 (Hutchens & Yip), each of which is incorporated herein by reference in its entirety. The two methods can be combined by, for example, using a SELDI affinity surface to capture an analyte and adding matrix-containing liquid to the captured analyte to provide the energy absorbing material.

In certain embodiments, the laser desorption/ionization mass spectrophotometer is further coupled to a quadrupole time-of-flight mass spectrometer QqTOF MS (see, e.g., Krutchinsky et al., WO 99/38185). Methods such as MALDI-QqTOFMS (Krutchinsky et al., WO 99/38185; Shevchenko et al. (2000) Anal. Chem. 72: 2132-2141), ESI-QqTOF MS (Figeys et al. (1998) Rapid Comm'ns. Mass Spec. 12-1435-144) and chip capillary electrophoresis (chip-CE)-QqTOF MS (Li et al. (2000) Anal. Chem. 72: 599-609) have been described previously.

In one embodiment, a mass spectrometer is used to fractionate protein samples of the invention. In a typical mass spectrometer, a substrate containing a protein analyte is introduced into an inlet system of the mass spectrometer. The analyte is then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a detector.

The detector then translates information of the detected ions into mass-to-charge ratios. Detection of the presence of a marker or other substances will typically involve detection of signal intensity. This, in turn, can reflect the quantity and character of a protein bound to the substrate. The mass spectrometers and their techniques are well known to those of skill in the art. Any person skilled in the art understands, any of the components of a mass spectrometer (e.g., desorption source, mass analyzer, detect, etc.) can be combined with other suitable components described herein or those known in the art. For additional information regarding mass spectrometers, see, e.g., Principles of Instrumental Analysis, 3rd ed., Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

In one embodiment, a laser desorption time-of-flight mass spectrometer is used with the substrate of the present invention. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

B. Chromatographic Separation Procedures

Any of a wide variety of chromatographic procedures may be employed according to the present invention to separate proteins. For example, thin layer chromatography, gas chromatography, high performance liquid chromatography, paper chromatography, affinity chromatography or supercritical flow chromatography may be used to effect separation of various chemical species.

Partition chromatography is based on the theory that if two phases are in contact with one another, and if one or both phases constitute a solute, the solute will distribute itself between the two phases. Usually, partition chromatography employs a column, which is filled with a sorbent and a solvent. The solution containing the solute is layered on top of the column. The solvent is then passed through the column, continuously, which permits movement of the solute through the column material. The solute can then be collected based on its movement rate. The two most common types of partition chromatograph are paper chromatograph and thin-layer chromatograph (TLC); together these are called adsorption chromatography. In both cases, the matrix contains a bound liquid. Other examples of partition chromatography are gas-liquid and gel chromatography.

Paper chromatography is a variant of partition chromatography that is performed on cellulose columns in the form of a paper sheet. Cellulose contains a large amount of bound water even when extensively dried. Partitioning occurs between the bound water and the developing solvent. Frequently, the solvent used is water. Usually, very small volumes of the solution mixture to be separated is placed at top of the paper and allowed to dry. Capillarity draws the solvent through the paper, dissolves the sample, and moves the components in the direction of flow. Paper chromatograms may be developed for either ascending or descending solvent flow. Two dimensional separations are permitted by changing the axis of migration 90° after the first run.

Thin layer chromatography (TLC) allows the use of any substance that can be finely divided and formed into a uniform layer. In TLC, the stationary phase is a layer of sorbent spread uniformly over the surface of a glass or plastic plate. The plates are usually made by forming a slurry of sorbent that is poured onto the surface of the gel after creating a well by placing tape at a selected height along the perimeter of the plate. After the sorbent dries, the tape is removed and the plate is treated just as paper in paper chromatography. The sample is applied and the plate is contacted with a solvent. Once the solvent has almost reached the end of the plate, the plate is removed and dried. Spots can then be identified by fluorescence, immunologic identification, counting of radioactivity, or by spraying varying reagents onto the surface to produce a color change.

In Gas-Liquid chromatography (GLC), the mobile phase is a gas and the stationary phase is a liquid adsorbed either to the inner surface of a tube or column or to a solid support. The liquid usually is applied as a solid dissolved in a volatile solvent such as ether. The sample, which may be any sample that can be volatized, is introduced as a liquid with an inert gas, such as helium, argon or nitrogen, and then heated. This gaseous mixture passes through the tubing. The vaporized compounds continually redistribute themselves between the gaseous mobile phase and the liquid stationary phase, according to their partition coefficients.

The advantage of GLC is in the separation of small molecules. Sensitivity and speed are quite good, with speeds that approach 1000 times that of standard liquid chromatography. By using a non-destructive detector, GLC can be used preparatively to purify grams quantities of material. The principal use of GLC has been in the separation of alcohols, esters, fatty acids and amines.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

The gel material for gel chromatography is a three-dimensional network whose structure is usually random. The gels consist of cross-linked polymers that are generally inert, do not bind or react with the material being analyzed, and are uncharged. The space filled within the gel is filled with liquid and this liquid occupies most of the gel volume. Common gels are dextran, agarose and polyacrylamide; they are used for aqueous solution.

High Performance Liquid Chromatography (HPLC) is characterized by a very rapid separation with extraordinary resolution of peaks. This is achieved by the use of very fine particles and high pressure to maintain and adequate flow rate. Separation can be accomplished in a matter of minutes, or a most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

C. Immunodetection Methods

In still further embodiments, once the proteins are separated, immunodetection methods can be used for binding, purifying, removing, quantifying and/or otherwise generally detecting biological components. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, 1999; Gulbis B and Galand P, 1993; De Jager R et al., 1993; and Nakamura et al., 1987, each incorporated herein by reference. For a general description of these techniques, see also Sambrook et al., Molecular Cloning, A Laboratory Manual (2nd ed. 1989); and Current Protocols in Molecular Biology (Ausubel et al., eds., 1994).

Thus, antibodies to certain proteins or markers can be used in characterizing the proteineous composition of the present invention (e.g., alpha-1 antitrypsin, apolipoprotein-a-1, transtheyretin, complement factor B, apolipoprotein A1, apolipoprotein B-100, macroglobulin A2, proteo cadherin gamma A6, haptoglobin fragment, haptoglobin precursor, serum amyloid A, fibrinogen A, fibrinogen B, fibrinogen G, annexin AI, complement C4, transferrin, etc.) through techniques such as ELISAs and Western blot analysis. Antibodies used in the present invention can be either obtained commercially or produced utilizing standard methods known to those of skill in the art. For a general description of these techniques, see also Sambrook et al., Molecular Cloning, A Laboratory Manual (2nd ed. 1989); and Current Protocols in Molecular Biology (Ausubel et al., eds., 1994).

The steps of various other useful immunodetection methods have been described in the scientific literature, such as, e.g., Nakamura et al., (1987). Immunoassays, in their most simple and direct sense, are binding assays. Certain preferred immunoassays are the various types of radioimmunoassays (RIA) and immunobead capture assay. Immunohistochemical detection using tissue sections also is particularly useful. However, it will be readily appreciated that detection is not limited to such techniques, and Western blotting, dot blotting, FACS analyses, and the like also may be used in connection with the present invention.

Antibody compositions of the proteins or markers of the present invention will find great use in immunoblot or Western blot analysis. The antibodies may be used as high-affinity primary reagents for the identification of proteins immobilized onto a solid support matrix, such as nitrocellulose, nylon or combinations thereof. In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody or a biotin/avidin ligand binding arrangement, as is known in the art.

1. Antibody Preparation

The antibodies of the present invention may be produced using standard procedures that are well known and used in the art. Yet further, commercial available antibodies can also be used.

a) Polyclonal Antibodies

Polyclonal antibodies are generally raised in animals by multiple subcutaneous (sc) or intraperitoneal (ip) injections of the protein (e.g., alpha-1 antitrypsin, apolipoprotein-a-1, transtheyretin, complement factor B, apolipoprotein A1, apolipoprotein B-100, macroglobulin A2, proteo cadherin gamma A6, haptoglobin fragment, haptoglobin precursor, serum amyloid A, fibrinogen A, fibrinogen B, fibrinogen G, annexin AI, complement C4, transferrin, etc.) and an adjuvant.

Animals are immunized against the immunogenic composition or derivatives. Animals are boosted until the titer plateaus. The animals are usually bled through an ear vein or alternatively by cardiac puncture. The removed blood is allowed to coagulate and then centrifuged to separate serum components from whole cells and blood clots. The serum may be used as is for various applications or else the desired antibody fraction may be purified by well-known methods, such as affinity chromatography using another antibody, a peptide bound to a solid matrix, or by using, e.g., protein A or protein G chromatography.

b) Monoclonal Antibodies

The methods for generating monoclonal antibodies (MAbs) generally begin along the same lines as those for preparing polyclonal antibodies. Rodents such as mice and rats are preferred animals, however, the use of rabbit, sheep, goat, monkey cells also is possible. The use of rats may provide certain advantages (Goding, 1986), but mice are preferred, with the BALB/c mouse being most preferred as this is most routinely used and generally gives a higher percentage of stable fusions.

Monoclonal antibodies are obtained from a population of substantially homogeneous antibodies, i.e., the individual antibodies comprising the population are identical except for possible naturally-occurring mutations that may be present in minor amounts. Thus, the modifier “monoclonal” indicates the character of the antibody as not being a mixture of discrete antibodies.

The animals are injected with antigen, generally as described above for polyclonal antibodies. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals.

Following immunization, somatic cells with the potential for producing antibodies, specifically B-lymphocytes (B cells), are selected for use in the MAb generating protocol. These cells may be obtained from biopsied spleens or lymph nodes. Spleen cells and lymph node cells are preferred, the former because they are a rich source of antibody-producing cells that are in the dividing plasmablast stage.

Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B-lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1976), and those using polyethylene glycol (PEG), such as 37% (v/v) PEG. The use of electrically induced fusion methods also is appropriate (Goding, 1986).

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide MAbs. The cell lines may be exploited for MAb production in two basic ways.

A sample of the hybridoma can be injected (often into the peritoneal cavity) into a histocompatible animal of the type that was used to provide the somatic and myeloma cells for the original fusion (e.g., a syngeneic mouse). Optionally, the animals are primed with a hydrocarbon, especially oils such as pristane (tetramethylpentadecane) prior to injection. The injected animal develops tumors secreting the specific monoclonal antibody produced by the fused cell hybrid. The body fluids of the animal, such as serum or ascites fluid, can then be tapped to provide MAbs in high concentration.

The individual cell lines could also be cultured in vitro, where the MAbs are naturally secreted into the culture medium from which they can be readily obtained in high concentrations.

MAbs produced by either means may be further purified, if desired, using filtration, centrifugation and various chromatographic methods such as HPLC or affinity chromatography. Fragments of the monoclonal antibodies of the invention can be obtained from the purified monoclonal antibodies by methods, which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, combinatorial immunoglobulin phagemid libraries are prepared from RNA isolated from the spleen of the immunized animal, and phagemids expressing appropriate antibodies are selected by panning using cells expressing the antigen and control cells e.g., normal-versus-tumor cells. The advantages of this approach over conventional hybridoma techniques are that approximately 10⁴ times as many antibodies can be produced and screened in a single round, and that new specificities are generated by H and L chain combination which further increases the chance of finding appropriate antibodies.

c) Humanized Antibodies

Methods for humanizing non-human antibodies are well known in the art. Generally, a humanized antibody has one or more amino acid residues introduced into it from a source, which is non-human. These non-human amino acid residues are often referred to as “import” residues, which are typically taken from an “import” variable domain. Humanization can be essentially performed following the method of Winter and co-workers (Jones et al., 1986; Riechmann et al., 1988; Verhoeyen et al., 1988), by substituting rodent CDRs or CDR sequences for the corresponding sequences of a human antibody. Accordingly, such “humanized” antibodies are chimeric antibodies wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

It is important that antibodies be humanized with retention of high affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e. the analysis of residues that influence the ability of the candidate immunoglobulin to bind its antigen.

d) Human Antibodies

Human monoclonal antibodies can be made by the hybridoma method. Human myeloma and mouse-human heteromyeloma cell lines for the production of human monoclonal antibodies have been described (Kozbor, 1984; U.S. Pat. No. 6,150,584, which is incorporated herein by reference).

It is now possible to produce transgenic animals (e.g., mice) that are capable, upon immunization, of producing a repertoire of human antibodies in the absence of endogenous immunoglobulin production. For example, it has been described that the homozygous deletion of the antibody heavy chain joining region (JH) gene in chimeric and germ-line mutant mice results in complete inhibition of endogenous antibody production. Transfer of the human germ-line immunoglobulin gene array in such germ-line mutant mice will result in the production of human antibodies upon antigen challenge. (Jakobovits et al., 1993).

Alternatively, the phage display technology (McCafferty et al., 1990) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle.

2. Western Blot Analysis

Western blot analysis is an established technique that is commonly employed for analyzing and identifying proteins. The proteins are first separated by electrophoresis in polyacrylamide gel, then transferred (“blotted”) onto a nitrocellulose membrane or treated paper, where they bind in the same pattern as they formed in the gel. The antigen is overlaid first with antibody, then with anti-immunoglobulin or protein A labeled with a radioisotope, fluorescent dye, or enzyme. One of ordinary skill in the art would be familiar with this commonly used technique for quantifying protein in a sample.

3. ELISAs

Immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

In one exemplary ELISA, the antibodies of the proteineous marker of the invention are immobilized onto a selected surface exhibiting protein affinity, such as a well in a polystyrene microtiter plate. Then, a test composition suspected of containing the proteineous marker, such as a clinical sample, is added to the wells. After binding and/or washing to remove non-specifically bound immune complexes, the bound proteineous marker protein antigen may be detected. Detection is generally achieved by the addition of another antibody that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the proteineous marker protein antigen are immobilized onto the well surface and/or then contacted with the antibodies of the proteineous marker of the invention. After binding and/or washing to remove non-specifically bound immune complexes, the bound anti-proteineous marker antibodies are detected. Where the initial anti-proteineous marker antibodies are linked to a detectable label, the immune complexes may be detected directly. Again, the immune complexes may be detected using a second antibody that has binding affinity for the first anti-proteineous marker antibody, with the second antibody being linked to a detectable label.

Another ELISA in which the proteineous marker proteins, polypeptides and/or peptides are immobilized, involves the use of antibody competition in the detection. In this ELISA, labeled antibodies against the proteineous marker are added to the wells, allowed to bind, and/or detected by means of their label. The amount of proteineous marker antigen in an unknown sample is then determined by mixing the sample with the labeled antibodies against the proteineous marker before and/or during incubation with coated wells. The presence of proteineous marker in the sample acts to reduce the amount of antibody against proteineous marker available for binding to the well and thus reduces the ultimate signal. This is also appropriate for detecting antibodies against a proteineous marker in an unknown sample, where the unlabeled antibodies bind to the antigen-coated wells and also reduces the amount of antigen available to bind the labeled antibodies.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

4. Immunohistochemistry

Antibodies of the protein or markers of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in 70° C. isopentane; cutting the plastic capsule and/or removing the frozen cylinder of tissue; securing the tissue cylinder on a cryostat microtome chuck; and/or cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections.

5. Immunoelectron Microscopy

The antibodies of the proteins or markers of the present invention may also be used in conjunction with electron microscopy to identify intracellular tissue components. Briefly, and electron-dense label is conjugated directly or indirectly to the anti-proteineous marker antibody. Examples of electron-dense labels according to the invention are ferritin and gold. The electron-dense label absorbs electrons and can be visualized by the electron microscope.

6. Protein Array Technology

Protein array technology allows high-throughput screening for gene expression and molecular interactions. Protein arrays appear as new and versatile tools in functional genomics, enabling the translation of gene expression patterns of normal and diseased tissues into protein product catalog. Protein function, such as enzyme activity, antibody specificity, and other ligand-receptor interactions and binding of nucleic acids or small molecules can be analyzed on a whole-genome level.

a) Protein Biochip Assays

These arrays, which contain thousands of different proteins or antibodies spotted onto glass slides or immobilized in tiny wells, allow one to examine the biochemical activities and binding profiles of a large number of proteins at once. To examine protein interactions with such an array, a labeled protein is incubated with each of the target proteins immobilized on the slide, and then one determines which of the many proteins the labeled molecule binds.

The basic construction of protein chips has some similarities to DNA chips, such as the use of a glass or plastic surface dotted with an array of molecules. These molecules can be DNA or antibodies that are designed to capture proteins. Defined quantities of proteins are immobilized on each spot, while retaining some activity of the protein. With fluorescent markers or other methods of detection revealing the spots that have captured these proteins, protein microarrays are being used as powerful tools in high-throughput proteomics and drug discovery.

Glass slides are still widely used, since they are inexpensive and compatible with standard microarrayer and detection equipment. However, their limitations include multiple-based reactions, high evaporation rates, and possible cross-contamination.

Matrix slides offer a number of advantages, such as reduced evaporation and no possibility of cross-contamination, but they are expensive. Nanochips for proteomics have the same advantages, in addition to reduced cost and the capability of multiple-component reactions.

The earliest and best-known protein chip is the ProteinChip by Ciphergen Biosystems Inc. (Fremont, Calif.). The ProteinChip is based on the surface-enhanced laser desorption and ionization (SELDI) process. Known proteins are analyzed using functional assays that are on the chip. For example, chip surfaces can contain enzymes, receptor proteins, or antibodies that enable researchers to conduct protein-protein interaction studies, ligand binding studies, or immunoassays. With state-of-the-art ion optic and laser optic technologies, the ProteinChip system detects proteins ranging from small peptides of less than 1000 Da up to proteins of 300 kDa and calculates the mass based on time-of-flight (TOF).

The ProteinChip biomarker system is the first protein biochip-based system that enables biomarker pattern recognition analysis to be done. This system allows researchers to address important clinical questions by investigating the proteome from a range of crude clinical samples (i.e., laser capture microdissected cells, biopsies, tissue, urine, and serum). The system also utilizes biomarker pattern software that automates pattern recognition-based statistical analysis methods to correlate protein expression patterns from clinical samples with disease phenotypes.

Some systems can perform biomarker discovery in days and validation of large sample sets within weeks. Its robotics system accessory automates sample processing, allowing hundreds of samples to be run per week and enabling a sufficient number of samples to be run, which provides high statistical confidence in comprehensive studies for marker discovery and validation.

b) Microfluidic Chip-Based Immunoassays

Microfluidics is one of the most important innovations in biochip technology. Since microfluidic chips can be combined with mass spectrometric analysis, a microfluidic device has been devised in which an electrospray interface to a mass spectrometer is integrated with a capillary electrophoresis channel, an injector, and a protein digestion bed on a monolithic substrate (Wang et al., 2000). This chip thus provides a convenient platform for automated sample processing in proteomics applications.

These chips can also analyze expression levels of serum proteins with detection limits comparable to commercial enzyme-linked immunosorbent assays, with the advantage that the required volume sample is markedly lower compared with conventional technologies.

Biosite (San Diego) manufactures the Triage protein chip that simultaneously measures 100 different proteins by immunoassays. The Triage protein chip immunoassays are performed in a microfluidic plastic chip, and the results are achieved in 15 minutes with picomolar sensitivities. Microfluidic fluid flow is controlled in the protein chip by the surface architecture and surface hydrophobicity in the microcapillaries. The immunoassays utilize high-affinity antibodies and a near-infrared fluorescent label, which is read by a fluorometer.

c) Tissue Microarray Technology

Tissue microarray technology provides a high-throughput approach for linking genes and gene products with normal and disease tissues at the cellular level in a parallel fashion. Compared with classical in situ technologies in molecular pathology that are very time-consuming, tissue microarrays provide increased throughput in two ways: up to 1000 tissue specimens can be analyzed in a single experiment, either at the DNA, RNA, or protein level; and tens of thousands of replicate tissue microarrays can be generated from a set of tissues. This process provides a template for analyzing many more biomarkers than has ever been possible previously in a clinical setting, even using archival, formalin-fixed specimens.

d) Nanoscale Protein Analysis

Most current protocols including protein purification and automated identification schemes yield low recoveries that limit the overall process in terms of sensitivity and speed. Such low protein yields and proteins that can only be isolated from limited source material (e.g., biopsies) can be subjected to nanoscale protein analysis: a nanocapture of specific proteins and complexes, and optimization of all subsequent sample-handling steps, leading to a mass analysis of peptide fragments. This focused approach, also termed targeted proteomics, involves examining subsets of the proteome (e.g., those proteins that are specifically modified, bind to a particular DNA sequence, or exist as members of higher-order complexes or any combination thereof). This approach is used to identify genetic determinants of cancer that alter cellular physiology and respond to agonists.

A new detection technique called multiphoton detection, by Biotrace Inc. (Cincinnati), can quantify subzeptomole amounts of proteins and will be used for diagnostic proteomics, particularly for cytokines and other low-abundance proteins. Biotrace is also developing supersensitive protein biochips to detect concentrations of proteins as low as 5 fg/ml (0.2 attomole/ml), thereby permitting sensitivity that is 1000 times greater than current protein biochips.

### III. Method of Diagnosing a Vessel Disorder

The acuteness or severity of the symptoms often dictates how rapidly a diagnosis must be established and treatment initiated. For example, immediate diagnosis and care of a patient experiencing a variety of acute conditions can be critical. See, e.g., Harris, Aust. Fam. Physician 31: 802-06 (2002) (asthma); Goldhaber, Eur. Respir. J. Suppl. 35:22s-27s (2002) (pulmonary embolism); Lundergan et al., Am. Heart J. 144: 456-62 (2002) (myocardial infarction). However, even in cases where the apparent symptoms appear relatively stable, rapid diagnosis, and the rapid initiation of treatment, can provide both relief from immediate discomfort and advantageous improvement in prognosis.

Thus, certain embodiments of the present invention relate to a method of diagnosing a subject suspected of having a vessel disorder. In diagnosing a subject suspected of having a vessel disorder, a biological sample, for example a blood sample, bodily fluid sample (e.g., saliva, etc) or tissue sample, is obtained from the subject. A protein profile is determined or measured in the sample using procedures described herein, for example, mass spectrometry, immunodetection (e.g., antibody binding, for example, ELISAs, Western blotting, etc). The protein profile from the subject is compared to a protein profile in a healthy control, wherein an alteration in the levels of a protein of the profile in the subject compared to the healthy control is indicative of a vessel disorder. In certain embodiments, alteration in the levels of a protein in the profile refers to the presence or absence of a protein, thus the diagnosis is based upon qualitative measurements. This does not exclude quantitative means. The protein profile of the present invention can also be used to determine and compare the amounts or levels of proteins within the profile.

The protein profile can contain one or more proteins having an atomic mass in the range of about 2,500 daltons to about 6,500 daltons; 6,000 daltons to about 12,000 daltons; 25,000 daltons to about 45,000 daltons; 45,000 daltons to about 65,000 daltons; 65,000 daltons to about 95,000 daltons; about 95,000 daltons to about 120,000 or any range therebetween. For example, the proteins can comprise an atomic mass in daltons of 1,171, 1,177, 1,384, 2,796, 2,796, 5,738, 5,761, 5,761, 6,380, 8,707, 8,707, 9,406, 9,406, 10,388, 10,388, 10,964, 10,977, 11,364, 11,410, 11,446, 11,497, 11,669, 11,743, 13,589, 16,000, 16,001, 19,459, 19,459, 20,000, 30,778, 31,358, 31,358, 45,177, 45,294, 45,294, 45,595, 45,595, 46,091, 46,091, 46,160, 46,707, 60,279, 67,174, 67,174, 67,307, 92,112, or 114,287 daltons. One of skill in the art further understands that the atomic mass in daltons includes in fraction of the masses indicated, for example, but not limited to, for example, 2796.0, 5763.0, 5816.8, 5837.7, 6380.3, 8097.0, 10390.5, 10983.5, 11074.5, 11423.1, 11649.1, 11828.1, 10983.5, 11649.1, 28391.3, 29212.7, 31358.4, 44792.5, 45313.3, 45595.8, 45937.1, 46005.0, 46160.6, 46182.4, 66549.9, 67251.0, 37320.3, 92112.6, 100901.8, and 114287.1 daltons.

Yet further, the protein profile can be one or more proteins, for example, but not limited to alpha-1 antitrypsin, transthyretin, apolipoprotein-a-1, serum amyloid A, haptoglobin, haptoglobin precursor, haptoglobin fragments, fibrinogen A, fibrinogen B, fibrinogen G, annexin AI, complement C4, complement factor B, apolipoprotein A1, apoliprotein B-100, macroglobulin A2, proteo cadherin gamma A6, transferrin, transferrin fragments, and a combination thereof. More specifically, the proteins of the protein profile can be proteolytic fragments that are present in the blood of the subject, for example, but not limited to haptoglobin fragments, transferrin, fibrinogen G, etc.

In certain embodiments, the vessel disorder is an aneurysm. Aneurysms are a pathologic dilation of a segment of a vessel. Aneurysms can occur in any type of blood vessels, however, they generally occur in an artery, for example, aorta, cerebral arteries, and peripheral arteries, such as those in the legs.

A “true” aneurysm involves all three layers of the vessel wall, which is different from a “pseudoaneurysm” that involves less than all the layers of the vessel, such as the intimal and medial layers are disrupted. Another type of aneurysm is a fusiform with affects the entire circumference of a segment of the vessel. Still further, another type of an aneurysm is saccular aneurysm which involves only a portion of the circumference, resulting in an outpouching of the vessel wall.

Another vessel disorder includes false aneurysm. Unlike a true aneurysm, this type of aneurysm does not involve the vessel wall. It merely represents a collection of blood, which has been held around the vessel by connective tissue. Such false aneurysms may arise following traumatic damage or there may be a slow anastomotic leak which is confined by surrounding tissues. False aneurysm result in a slowly expanding blood-filled cavity. The cavity will eventually rupture or undergo thrombosis.

Aneuysmal disorders or aneurysmal diseases include those processes that result in aneurysm formation in a segment of at least one artery. An aneurysm is considered a permanent localized dilatation of an artery with increase in diameter of 1.5 times normal, recognizing that values for normal arterial diameter vary with age, sex and blood pressure. (Vermilion B D et al., 1981). Aneurysms cause symptoms by rupturing, by compressing adjacent structures, by leaking, by obstructing flow to vessels that arise from the segment of artery affected by the aneurysm, and by accumulating thrombus within the vessel lumen at the site of the aneurysm, said thrombus being capable of suddenly occluding the vessel or of sending smaller emboli into the distal arterial tree.

Aneurysms may be classified according to the anatomic site where they arise. Common types of aneurysms include aortic aneurysms, peripheral aneurysms, visceral aneurysms and intracranial aneurysms. The most common location for anarterial aneurysm is the infrarenal aorta. Aneurysms of the infrarenal aorta, also referred to as abdominal aortic aneurysms or “AAAs,” occur three to seven times more frequently than thoracic aneurysms. Aneurysms at other anatomic locations are frequently found in patients with AAAs, locations including the common or internal iliac artery and the femoral-popliteal arterial system. (Goldstone J, 1998). So-called peripheral aneurysms involve those arteries of the upper extremities distal to and including the subclavian artery, those arteries of the lower extremities distal to and including the femoral artery, and the extracranial carotid arteries. Peripheral aneurysms are rare as compared to aortic aneurysms. Unlike AAAs, which tend to rupture, peripheral aneurysms tend to thrombose or give rise to arterial aneurysms. (Flanigan D, 1998). Unlike AAAs, where size is a main determinant of the need for surgery, peripheral aneurysms tend to be corrected surgically as soon as they are identified. Visceral aneurysms involve the major branches of the abdominal aorta. (Stanley J C et al., 1998). These rare types of aneurysms arise from the splanchnic vessels and the renal arteries. About one fourth of splanchnic artery aneurysms present as acute surgical emergencies, with a high rate of mortality. Intracranial aneurysms are typically congenital in origin, evolving and expanding during life. (Hoff J T et al., 1999). Typically they are found at the bifurcations of the major vessels of the arterial Circle of Willis. Up to 20% of patients with intracranial aneurysms have multiple aneurysms. The most common presentation for an intracranial aneurysm is subarachnoid hemorrhage, with varying degrees of severity that can extend to permanent neurological deficit, coma and death.

Most subjects are unaware that they have an aneurysm because in most cases, there are no symptoms. Thus, the present invention can be used to identify those patients suspected of having an aneurysm such that they can receive proper treatment prior to the aneurysm rupturing.

Yet further, the present invention can be used to diagnose a subject suspected or at risk or for having a dissection. Dissections are a tear in the intima of a vessel. The tear can be circumferential or transverse. The most prominate dissections are aortic dissections. Acute aortic dissections occur when the blood from the lumen of the aorta dissects into the wall of the aorta. Acute dissections are divided based on the anatomical location of the tear in the aorta with type A dissection initiating in the ascending aorta distal to aortic valve and type B dissections initiating in the descending aorta distal to the take off of the subclavian artery. A type A dissection is the most common type of dissections and is a medical emergency with 80% mortality in the first 48 hours. A type B dissection is also a medical emergency but the mortality is lower. An acute dissection is difficult to diagnose in the emergency room with many individuals sent home with undiagnosed dissections, and the end result is premature death. The inventors sought to identify a biomarker of an acute dissection that could be used in the ER setting to aid in the correct diagnosis and treatment of individuals with an acute dissection.

Thus, the present invention can be used as a diagnostic tool to diagnosing a subject suffering from or at risk of developing an aortic dissection. In diagnosing a subject suspected of having an aortic dissection, a biological sample, for example a blood sample, is obtained from the subject. A protein profile is determined or measured in the sample using procedures described herein, for example, mass spectrometry or immunodetection (e.g., ELISAs or Western blotting). The protein profile from the subject is compared to a protein profile in a healthy control, wherein an alteration in the levels of a protein of the profile in the subject compared to the healthy control is indicative of a aortic dissection.

The protein profile can contain one or more proteins having an atomic mass in the range of 2,500 daltons to about 6,500 daltons; 6,000 daltons to about 12,000 daltons; 25,000 daltons to about 45,000 daltons; 45,000 daltons to about 65,000 daltons; 65,000 daltons to about 95,000 daltons; or about 95,000 daltons to about 120,000. In certain embodiments, it is envisioned that the amounts or levels of these proteins will be enhanced or more prevalent in a subject having an aortic dissection compared to controls or non-diseased subjects. The analysis is performed on a statistical basis followed by a visual inspection of the data. The statistical measure is a p value <0.05 as significant differences between the two populations. Alternatively, the protein profile is measured qualitative. For example, alteration in the levels of a protein in the profile refers to the presence or absence of a protein, thus the diagnosis is based upon qualitative measurements. In certain embodiments, diagnosis is determined upon a fold difference or fold increase in the amount of at least one protein. The fold difference can be for example, at least a 2-fold increase, at least a 3-fold increase, at least a 4-fold increase, at least a 5-fold increase, at least a 6-fold increase, at least a 7-fold increase, at least a 8-fold increase, at least a 9-fold increase, at least a 10-fold increase, at least a 11-fold increase, at least a 12-fold increase, at least a 13-fold increase, at least a 14-fold increase, at least a 15-fold increase, at least a 20-fold increase, at least a 25-fold increase, at least a 30-fold increase, at least a 35-fold increase, at least a 40-fold increase, at least a 45-fold increase, at least a 50-fold increase, at least a 60-fold increase, at least a 70-fold increase, at least a 80-fold increase, at least a 90-fold increase, at least a 100-fold increase or any range therebetween.

A protein profile that is elevated or prevalent in controls or non-diseased subjects can comprise one or more proteins having an atomic mass in the range of about 6,400 to about 6,500; about 13,900 to about 14,400; about 8,900 to about 9,400 and about 16,600 to about 17,100. Thus, an indication that these markers or proteins are elevated in a subject would indicate that the subject did not have an aortic dissection.

Generally, the protein profile for Type A can include proteins having an atomic mass in daltons in the range of about of 5,800 to about 5,900, about 8,136, and about 17,000 to about 17, 400 daltons. More particularly, the protein profile to diagnose a Type A dissection comprises one or more proteins having an atomic mass in daltons selected from the group consisting of 2,796, 5,738, 5,761, 6,380, 8,707, 9,406, 10,388, 11,364, 11,410, 11,446, 11,497, 11,669, 11,743, 19,459, 31,358, 45,294, 45,595, 46,091, 46,160, 67,174, 67,307, 92,112, 114,287. More specifically, the atomic mass of the protein can include, for example, 2796.0, 5,763.0, 5,816.8, 5,837.7, 6,380.3, 8,097.0, 10,983.5, 11,074.5, 11,423.1, 11,649.1, 11,828.1, 28,391.3, 29,212.7, 31,358.4, 44,792.5, 45,313.3, 45,595.8, 46,160.6, 67,251.0, 67,320.3, 100,901.8, 114,287.1 daltons or any range therebetween.

Generally, the protein profile for Type B can include proteins having an atomic mass in the range of about of 3,570 to about 3,590 daltons. More particularly, the protein profile that can be used to diagnose a Type B dissection comprises one or more proteins having an atomic mass in daltons selected from the group consisting of 92,112, 1,171, 1,177, 1,384, 2,796, 5,761, 8,707, 9,406, 10,388, 10,964, 10,977, 19,459, 31,358, 45,294, 45,595, 46,091, 60,279, 67,174. More specifically, the atomic mass of the protein can include, for example, 10,390.5, 10,983.5, 11,649.1, 11,828.1, 31,358.4, 44,792.5, 45,313.3, 45,937.1, 46,005.0, 46,160.6, 46,182.4, 66,549.9, 67,251.0, 67,320.3, 92,112.6, 114,287.1 daltons or any range therebetween.

Another embodiment of the present invention is a method of monitoring a subject having or suspected of having a vessel disorder comprising the steps of: obtaining a first sample from the subject at a first selected time point; measuring in the first sample a protein profile; obtaining a second sample from the subject at a second selected time point; measuring in the second sample a protein profile; and comparing the protein profile obtained from first sample and the second sample to assess the change in the profile between the first selected time point and the second selected time point.

### IV. Kits

Any of the compositions as identified in the present invention, such as a biomarker or protein profile or protein panels, described herein may be comprised in a kit. In a non-limiting example, a biomarker, and/or additional agent, may be comprised in a kit. The kits will thus comprise, in suitable container means, a biomaker and/or an additional agent of the present invention.

The kits may comprise a suitably aliquoted biomarker and/or additional agent compositions of the present invention, whether labeled or unlabeled, as may be used to prepare a standard curve for a detection assay. In certain embodiments, the kit may contain an antibody or antibody to the protein or proteins in the protein profile. Such antibodies can include, but are not limited to alpha-1 antitrypsin antibody, transthyretin antibody, apolipoprotein-a-1 antibody, serum amyloid A antibody, haptoglobin antibody, haptoglobin precursor antibody, haptoglobin antibody fragments, fibrinogen A antibody, fibrinogen B antibody, fibrinogen G antibody, annexin AI antibody, complement C4 antibody, complement factor B antibody, apolipoprotein A1 antibody, apoliprotein B-100 antibody, macroglobulin A2 antibody, proteo cadherin gamma A6 antibody, transferrin antibody, transferrin antibody fragments, and a combination thereof.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the biomarker, additional agent, and any other reagent containers in close confinement for commercial sale. Such containers may include injection or blow molded plastic containers into which the desired vials are retained.

Thus, certain embodiments of the present invention provides a kit for detecting a vessel disorder comprising: a marker that specifically detects a vessel disorder. More particularly, the a kit comprises at least two different containers, wherein a first container comprises a protein profile to a diseased subject. The second container contains a protein profile to a non-diseased subject. The kit may be used to assess the susceptibility of an subject or to diagnose a subject. For example, a biological sample is obtained from a subject to be tested, after the sample is obtained, the sample is incubated with the biomarker of the kit. The biomarkers contained in the kit can be analyzed by determining the presence of the labeled biomarker. Still further, other analysis can be used for example, mass spectrometry, etc.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Protein/Peptide Profiling of Plasma Samples

Plasma samples were obtained from 16 individuals presenting with acute dissections, 9 individuals with type A dissections and 7 individuals with type B dissections, along with 10 control individuals with dissection.

### Example 2

### Protein/Peptide Profiling of Plasma Samples

Analysis of plasma from control subjects or patients with acute aortic dissections was done using SELDI-TOF (Surface Enhanced Laser Desorption/Ionization-Time of Flight) mass spectrometry on a Ciphergen PBSII mass spectrometer. The major advantage in SELDI applications lies in the properties of the chips used for protein analysis. In SELDI applications the same protein mixture is applied to a series of spots, however chips have activated surfaces that permit the selective chemical desorption of the protein mixture. Profiling on the basis of isoelectric point on a weak cation exchange surface (WCX2 chip) was performed by dilution of a plasma sample into a solution buffered to pH 4, 6, 8, or 10. The buffered plasma sample is applied to a spot on the chip. After an appropriate incubation period with vigorous agitation, the plasma is removed and the spots washed extensively with the dilution buffer. The washed spots were treated with an energy absorbing matrix, sinnipinic acid and analyzed in a Ciphergen PBSII protein chip reader. This preliminary separation enabled the visualization and comparison of similar plasma protein fractions in different plasma samples and thus the identification of unique protein species in a particular sample population.

TABLE 1
Proteins/peptides identified by atomic mass

| Mol. Wt. | Dissection | Chip | Binding pH |
|---|---|---|---|
| 2,796 | Type A | WCX2 | 4 |
| 5,761 | Type A | WCX2 | 4 |
| 5,738 | Type A | WCX2 | 4 |
| 11,410 | Type A | WCX2 | 4 |
| 11,364 | Type A | WCX2 | 4 |
| 11,446 | Type A | WCX2 | 4 |
| 11,497 | Type A | WCX2 | 4 |
| 11,669 | Type A | WCX2 | 4 |
| 11,743 | Type A | WCX2 | 4 |
| 46,160 | Type A | WCX2 | 4 |
| 67,174 | Type A | WCX2 | 4 |
| 114,287 | Type A | WCX2 | 4 |
| 8,707 | Type A | WCX2 | 6 |
| 9,406 | Type A | WCX2 | 6 |
| 19,459 | Type A | WCX2 | 6 |
| 31,358 | Type A | WCX2 | 6 |
| 45,294 | Type A | WCX2 | 6 |
| 45,595 | Type A | WCX2 | 6 |
| 46,091 | Type A | WCX2 | 6 |
| 92,112 | Type A | WCX2 | 6 |
| 6,380 | Type A | WCX2 | 8 |
| 10,388 | Type A | WCX2 | 8 |
| 67,307 | Type A | WCX2 | 8 |
| 2,796 | Type B | WCX2 | 4 |
| 5,761 | Type B | WCX2 | 4 |
| 10,977 | Type B | WCX2 | 4 |
| 67,174 | Type B | WCX2 | 4 |
| 8,707 | Type B | WCX2 | 6 |
| 9,406 | Type B | WCX2 | 6 |
| 10,964 | Type B | WCX2 | 6 |
| 19,459 | Type B | WCX2 | 6 |
| 31,358 | Type B | WCX2 | 6 |
| 45,294 | Type B | WCX2 | 6 |
| 45,595 | Type B | WCX2 | 6 |
| 46,091 | Type B | WCX2 | 6 |
| 60,279 | Type B | WCX2 | 6 |
| 92,112 | Type B | WCX2 | 6 |
| 1,171 | Type B | WCX2 | 8 |
| 1,384 | Type B | WCX2 | 8 |
| 1,177 | Type B | WCX2 | 8 |
| 10,388 | Type B | WCX2 | 8 |

### Example 3

### Protein/Peptide Identification

Protein identification was achieved by the established methods of an initial purification by standard chromatographic procedures on the basis of molecular radius, isoelectric point or other physicochemical properties. The final purification was by polyacrylamide gel electrophoresis alone or in combination with isoelectric focusing on 2-dimensional gels. Protein spots were visualized by either Coomassie blue or silver staining procedures. Fractions from control and affected plasma samples were run under parallel conditions for a final demonstration of differential concentrations in the samples. Spots corresponding to candidate proteins/peptides were cut from the gel and treated with trypsin for in gel digestion. The peptide fragments were extracted and separated by capillary chromatography. The eluent was continuously monitored by electrospray ionization on an Applied Biosystems QStar XL LC/MS/MS Q-TOF mass spectrometer. The mass spectrometer was under an information dependent acquisition protocol that surveys the eluent for peptides and automatically subjects them to MS/MS analysis for peptide sequence information. Proteins were identified using the MASCOT search engine and the SwissProtein database.

TABLE 2

| Mass | Biomarker | Comment |
|---|---|---|
| 46,707 | Control | Marker is elevated in controls |
| | | relative to dissection |
| 16,001 | Control | Marker is elevated in controls |
| | | relative to dissection |
| 30,778 | Control | Marker is elevated in controls |
| | | relative to dissection |
| 13,589 | Type A, (Type B ?) | Elevated compared to control |
| 45,177 | Control | Marker is elevated in controls |
| | | relative to dissection |
| ~16,000 | Control | Two fragments tentatively |
| ~20,000 | Type A, Type B | from haptoglobulin of different |
| | | molecular weights. Controls |
| | | have a smaller fragment |
| | | compared to dissections |

### Example 4

### Immunodetection Protein/Peptide Identification

The following proteins were identified on the basis of partial chromatographic purification followed by SDS-PAGE. The gels were analyzed by scanning and a visual comparison of protein bands in plasma samples from control, Type A and Type B dissection patients. Bands were then excised from the gel and trypsinized. The resulting peptides in the trypsin digest were analyzed by LCMSMS for protein identification. The identification was based upon at least two peptides being identified with high levels of confidence and according to suggested guidelines from an NIH sponsored panel on Proteomics and Biomarker Discovery.

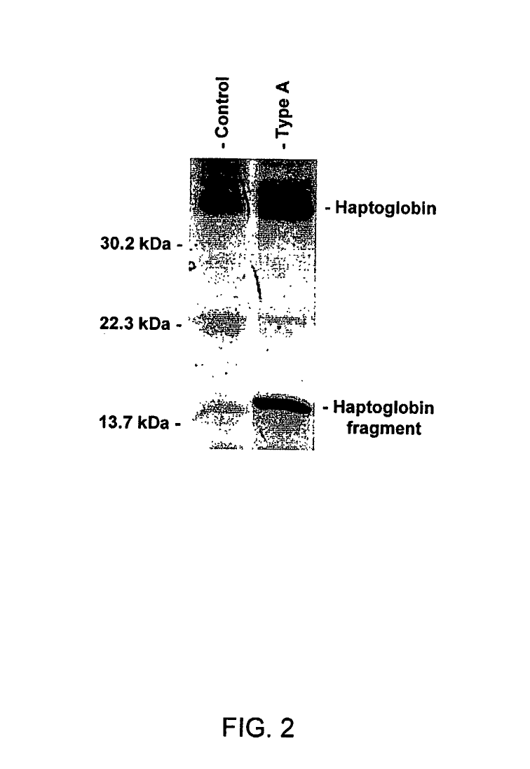

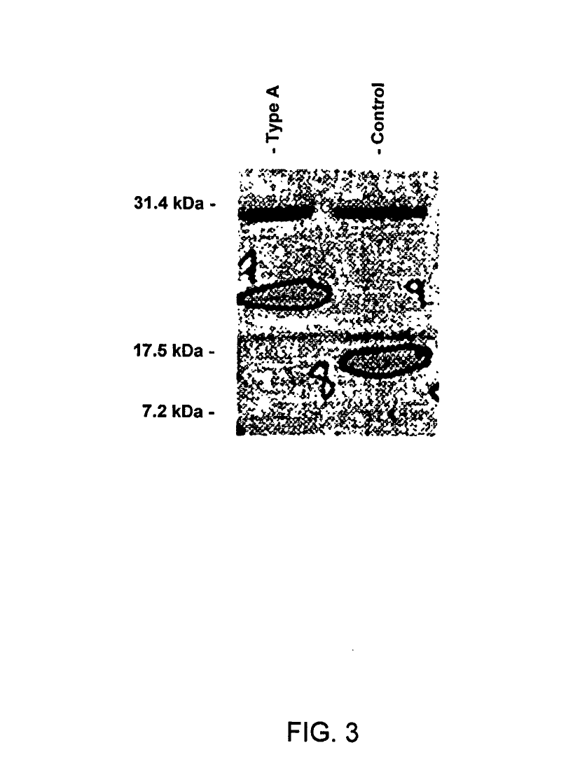

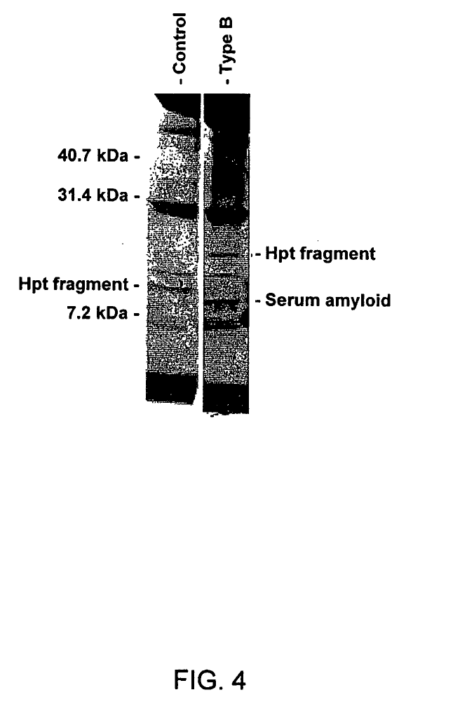

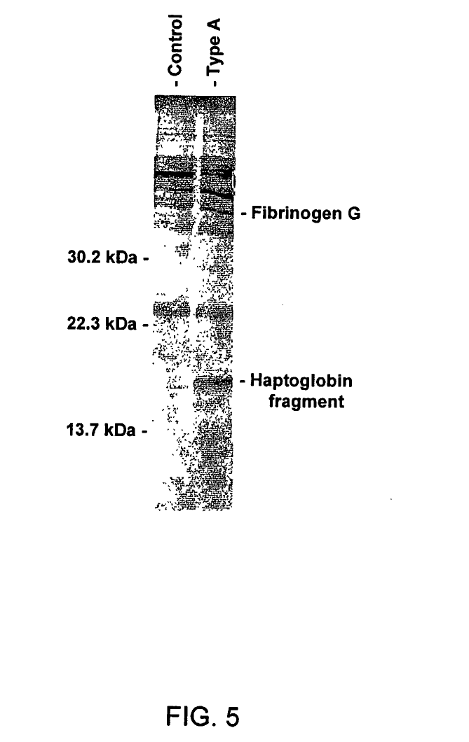

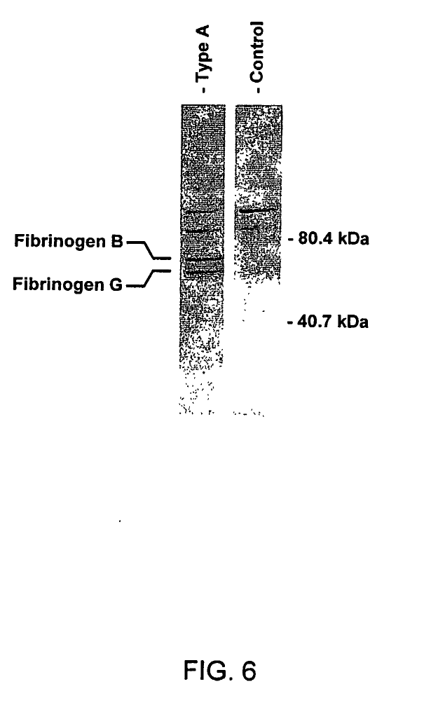

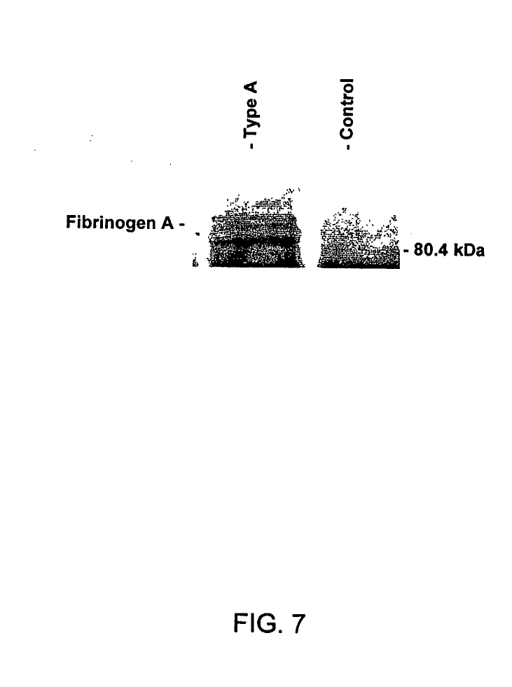

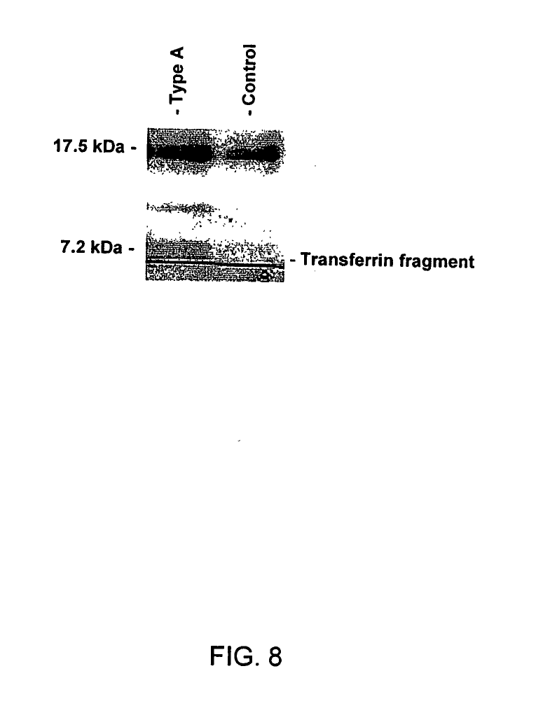

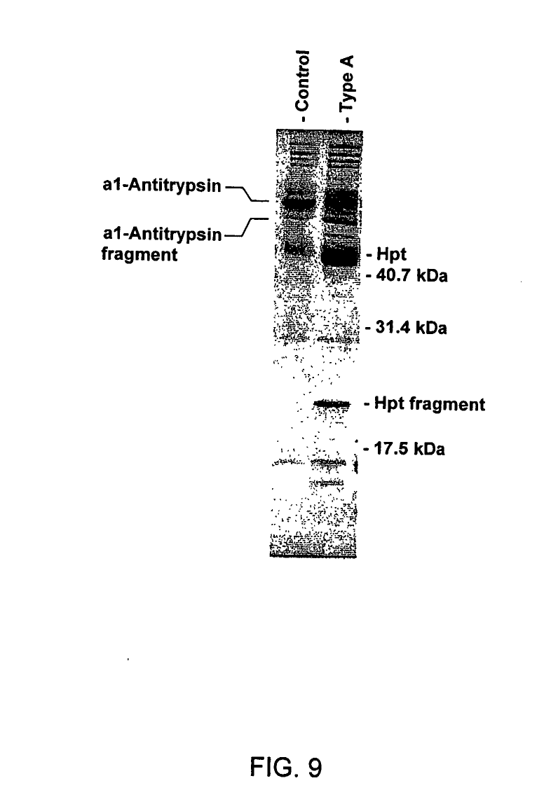

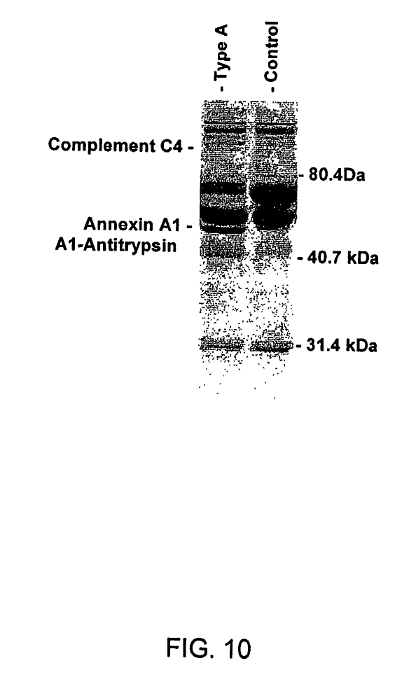

TABLE 3

| | Mass | | | |
|---|---|---|---|---|
| Protein | (Da) | Biomarker | Comment | Figure |
| Alpha-1 | 46,707 | Control | Marker is elevated in | FIG. 9 |
| antitrypsin | | | controls relative to | |
| | | | dissection | |
| Alpha-1 | ~43,000 | Type A | Marker is elevated in | FIGS. 9 |
| antitrypsin | | | controls relative to | and 10 |
| | | | dissection | |
| Serum amyloid A | 13,589 | Type A, | Elevated compared to | FIG. 4 |
| | | Type B | control | |
| Haptoglobin | 38,427 | Type A | Elevated compared to | FIGS. 2 |
| precursor | | | control levels | and 9 |
| Haptoglobin | ~15,000 | Control | A fragment of | FIGS. 3 |
| fragment | | | haptoglobin is found in | and 4 |
| | | | control plasma samples | |
| | | | that has an apparent | |
| | | | molecular weight from | |
| | | | SDS-PAGE of | |
| | | | approximately 15,000 Da | |
| Haptoglobin | ~19,000 | Type A, | A fragment of | FIGS. |
| fragment | | Type B | haptoglobin is found in | 2, 3, 4, 5 and |
| | | | Type A and Type B | 9 |
| | | | plasma samples that has | |
| | | | an apparent molecular | |
| | | | weight from SDS-PAGE | |
| | | | of approximately 19,000 | |
| | | | Da | |
| Fibrinogen A | 94,914 | Type A | Elevated in patient | FIG. 7 |
| Fibrinogen B | 55,892 | Type A | Elevated in Type A | FIG. 6 |
| | | | compared to control, full | |
| | | | length and fragments | |
| Fibrinogen G | 51,479 | Type A | Fragments elevated in | FIGS. 5 |
| | | | Type A | and 6 |
| Annexin AI | 38,559 | Type A | Elevated compared to | FIG. 10 |
| | | | control | |
| Complement C4 | 85,471 | Type A | Elevated in Type A | FIG. 10 |
| Transferrin | | Type A | Fragments running at very | FIG. 8 |
| | | | low molecular weight in | |
| | | | Type A | |

### Example 5

### Protein/Peptide Identification

The following have been identified by molecular weight on Ciphergen CHIPs and were separated on the basis of isoelectric point WCX2 weak cation exchange protein chips. Identification of these proteins was based upon a statistical analysis of mass spectra obtained on 10 control plasma samples, 13 Type A and 13 Type B dissection patient plasma samples. Tables 4A and 4B illustrate the intensity, which is the area under the peak. The values shown are for the intensity is the arithmetic mean of the areas of the peak in all of the samples in the group. The first selection criterion used to identify these peaks were that their intensity was greater than control with a p<0.05. The second selection criterion was based on visual inspection of the spectra revealing a readily identifiable peak compared to background noise or a readily identifiable alteration in the intensity of the peak compared to control.

TABLE 4A

| Type A | | Control | | Type A | SD - | |
|---|---|---|---|---|---|---|
| Dissection | | Intensity | SD - | Intensity | Type | Fold |
| M/Z Units? | p | (a.m) | Control | (a.m.) | A | Change |

WCX2 CHIP, pH 4

| | | | | | | |
|---|---|---|---|---|---|---|
| 10983.5 | 0.0002 | 0.119 | 0.053 | 1.181 | 0.948 | 9.92 |
| 11074.5 | 0.0077 | 0.145 | 0.067 | 1.397 | 1.504 | 9.63 |
| 11649.1 | 0.0110 | 1.041 | 0.949 | 4.033 | 3.146 | 3.87 |
| 11828.1 | 0.0156 | 0.577 | 0.541 | 1.571 | 1.460 | 2.72 |
| 11423.1 | 0.0407 | 0.297 | 0.283 | 6.879 | 7.699 | 23.1 |
| 46160.6 | 0.0156 | 0.254 | 0.188 | 0.509 | 0.210 | 2.00 |
| 114287.1 | 0.0029 | 0.012 | 0.012 | 0.037 | 0.020 | 3.08 |
| 100901.8 | 0.0131 | 0.083 | 0.080 | 0.191 | 0.119 | 2.30 |
| 5763.0 | 0.0004 | 0.015 | 0.078 | 0.948 | 0.568 | 63.2 |
| 5816.8 | 0.0005 | 0.186 | 0.203 | 2.294 | 2.209 | 12.3 |
| 5837.7 | 0.0014 | 0.169 | 0.149 | 2.007 | 1.509 | 11.9 |
| 2796.0 | 0.0019 | 0.284 | 0.460 | 2.569 | 2.247 | 9.04 |

WCX2 CHIP, pH 6

| | | | | | | |
|---|---|---|---|---|---|---|
| 8097.0 | 0.0024 | 0.996 | 0.694 | 0.315 | 0.359 | .316 |
| 28391.3 | 0.0013 | 0.171 | 0.051 | 0.362 | 0.105 | 2.12 |
| 29212.7 | 0.0013 | 0.098 | 0.034 | 0.250 | 0.193 | 2.55 |
| 44792.5 | 0.0031 | 0.210 | 0.104 | 0.610 | 0.310 | 2.90 |
| 45313.3 | 0.0112 | 0.146 | 0.085 | 0.503 | 0.377 | 3.45 |
| 31358.4 | 0.0142 | 0.026 | 0.028 | 0.121 | 0.121 | 4.65 |
| 45595.8 | 0.0346 | 0.155 | 0.097 | 0.471 | 0.392 | 3.04 |
| 67251.0 | 0.0142 | 0.329 | 0.220 | 1.122 | 0.996 | 3.41 |

WCX2 CHIP, pH 8

| | | | | | | |
|---|---|---|---|---|---|---|
| 6380.3 | 0.0071 | 0.056 | 0.108 | 1.803 | 4.302 | 32.2 |
| 67320.3 | 0.0046 | 0.164 | 0.048 | 0.402 | 0.228 | 2.45 |

TABLE 4B

| Type B | | Control | | Type B | SD - | |
|---|---|---|---|---|---|---|
| Dissection | | Intensity | SD - | Intensity | Type | Fold |
| M/Z Units? | p | (a.m.) | Control | (a.m.) | B | change |

WCX2 Chip, pH 4

| | | | | | | |
|---|---|---|---|---|---|---|
| 10983.5 | 0.0024 | 0.119 | 0.053 | 0.857 | 0.912 | 7.20 |
| 11649.1 | 0.0256 | 1.041 | 0.949 | 2.187 | 1.496 | 2.10 |
| 11828.1 | 0.0300 | 0.577 | 0.541 | 2.548 | 2.982 | 4.42 |
| 46160.6 | 0.0131 | 0.254 | 0.188 | 0.781 | 0.592 | 3.07 |
| 45937.1 | 0.0184 | 0.338 | 0.277 | 0.813 | 0.579 | 2.41 |
| 114287.1 | 0.0184 | 0.012 | 0.012 | 0.052 | 0.049 | 4.33 |

WCX2 Chip, pH 6

| | | | | | | |
|---|---|---|---|---|---|---|
| 31358.4 | 0.0058 | 0.026 | 0.028 | 0.188 | 0.238 | 7.23 |
| 45313.3 | 0.0138 | 0.146 | 0.085 | 0.692 | 0.719 | 4.74 |
| 44792.5 | 0.0207 | 0.210 | 0.104 | 0.644 | 0.566 | 3.07 |
| 46182.4 | 0.0207 | 0.100 | 0.077 | 0.605 | 0.660 | 6.05 |
| 46005.0 | 0.0367 | 0.126 | 0.088 | 0.635 | 0.669 | 5.04 |
| 92112.6 | 0.0170 | 0.083 | 0.084 | 0.358 | 0.340 | 4.31 |
| 67251.0 | 0.0252 | 0.329 | 0.220 | 0.713 | 0.480 | 2.17 |

WCX2 Chip, pH 8

| | | | | | | |
|---|---|---|---|---|---|---|
| 10390.5 | 0.0404 | 0.213 | 0.179 | 0.981 | 1.402 | 4.61 |
| 67320.3 | 0.0046 | 0.164 | 0.048 | 0.402 | 0.228 | 2.45 |
| 66549.9 | 0.0069 | 0.243 | 0.073 | 0.518 | 0.329 | 2.12 |

### REFERENCES CITED

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
Ausubel et al., Current Protocols in Molecular Biology, 1994).
Blackstock & Weir, Trends in Biotech. 17:121-127 (1999)
Dutt & Lee, Biochemical Engineering, pages 176-179 (April 2000)
Easton J D et al., pp. 2325-2348, in Harrison's.
Figeys et al. (1998) Rapid Comm'ns. Mass Spec. 12-1435-144
Figeys et al. (1998) Rapid Comm'ns. Mass Spec. 12-1435-144
Flanigan D, pp. 457-467 in W S Moore, Vascular Surgery: A Comprehensive Review, W B Saunders, 1998.
Goldstone J, pp. 435-455 in W S Moore, Vascular Surgery: A Comprehensive Review, W B Saunders, 1998.
Gygi et al., Nature Biotechnology 17:994-999 (1999)
Hoff J T et al., pp. 1877-1908 in Schwartz, S. I. (Ed.), Principles of Surgery, 7 th edition, McGraw-Hill, NY, 1999.
Li et al. (2000) Anal. Chem. 72: 599-609.
Mann, Nature Biotechnology 17:954-955 (1999)
Methods in Enzymology, Vol. 193: “Mass Spectrometry” (J. A. McCloskey, editor), 1990, Academic Press, New York.
Nankodo, K. K. Common Disease Series 4: Angina Pectoris.multidot.Myocardial Infarction, pp. 310-313.
Page et al., Drug Discovery Today 4:55-62 (1999)
Pandey & Mann, Nature 405:837-846 (2000).
Pleumeekers H J, et al., Int J Epidemiol, 28(4):682-6 1999.
Regnier et al., Trends in Biotech. 17:101-106 (1999)
Sambrook et al., Molecular Cloning, A Laboratory Manual (2nd ed. 1989)
Shevchenko et al. (2000) Anal. Chem. 72: 2132-2141
Stanley J C et al., pp. 468-481 in W S Moore, Vascular Surgery: A Comprehensive Review, W B Saunders, 1998.
Vermilion B D et al., Surgery 90:1009, 1981.
Wang & Hewick, Drug Discovery Today 4:129-133 (1999)
U.S. Pat. No. 5,118,937
U.S. Pat. No. 5,045,694
U.S. Pat. No. 5,719,060
U.S. Pat. No. 5,6020208
WO 98/59360
WO 98/59361
WO 98/59362
WO 99/38185
WO 99/38185

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A protein profile, said profile is indicative of a vessel disorder in a subject and comprises altered amounts of one or more proteins selected from the group consisting of alpha-1 antitrypsin, transthyretin, apolipoprotein-a-1, serum amyloid A, haptoglobin, haptoglobin precursor, haptoglobin fragments, fibrinogen A, fibrinogen B, fibrinogen G, annexin AI, complement C4, complement factor B, apolipoprotein A1, apoliprotein B-100, macroglobulin A2, proteo cadherin gamma A6, transferrin, transferrin fragments, and a combination thereof.

2. A protein profile, said profile is indicative of a vessel disorder in a subject and comprises altered amounts of one or more proteins having an atomic mass in the range of about 2,500 daltons to about 120,000 daltons.

3. The profile of claim 1 or 2, wherein the vessel disorder is selected from the group consisting of dissection, aneurysm, inflammatory disease, pseudoaneurysm, and false aneurysm.

4. The profile of claim 3, wherein the dissection is Type A or Type B.

5. The profile of claim 4, wherein said profile is indicative of a Type A aortic dissection in a subject and comprises altered amounts of one or more proteins having an atomic mass in daltons selected from the group consisting of 2796.0, 5,763.0, 5,816.8, 5,837.7, 6,380.3, 8,097.0, 10,983.5, 11,074.5, 11,423.1, 11,649.1, 11,828.1, 28,391.3, 29,212.7, 3,1358.4, 44,792.5, 45,313.3, 45,595.8, 46,160.6, 67,251.0, 67,320.3, 100,901.8, 114,287.1, and any combination thereof.

6. The protein profile of claim 4, wherein said profile is indicative of a Type B aortic dissection in a subject and comprises altered amounts of one or more proteins having an atomic mass in daltons selected from the group consisting of 10390.5, 10983.5, 11649.1, 11828.1, 31358.4, 44792.5, 45313.3, 45937.1, 46005.0, 46160.6, 46182.4, 66549.9, 67251.0, 67320.3, 92112.6, 114287.1, and any combination thereof.

7. The profile of claim 4, wherein said profile is indicative of a Type A aortic dissection in the subject and comprises altered amounts of one or more proteins selected from the group consisting alpha-1 antitrypsin, serum amyloid A, haptoglobin precursor, haptoglobin fragment, fibrinogen A, fibrinogen B, fibrinogen G, annexin AI, complement C4, transferrin and a combination thereof.

8. The profile of claim 4, wherein said profile is indicative of a Type B aortic dissection in the subject and comprises altered amounts of one or more proteins selected from the group consisting of serum amyloid A, haptoglobin, and a combination thereof.

9. A method of diagnosing a subject having a vessel disorder comprising the steps of:
obtaining a blood sample from the subject;
determining the protein profile of claim 1 or claim 2 in the sample; and
comparing the protein profile of the same proteins in a healthy control, wherein an alteration in the levels of a protein of the profile in the subject compared to the healthy control is indicative of a vessel disorder.

10. The method of claim 9, wherein the determining step comprises utilizing mass spectrometry or immunodetection.

11. The method of claim 10, wherein the immunodetection comprises antibody binding.

12. A method of monitoring a subject having or suspected of having a vessel disorder comprising the steps of:
obtaining a first blood sample from the subject at a first selected time point;
determining a protein profile in the first sample;
obtaining a second blood sample from the subject at a second selected time point;
determining a protein profile in the second sample;
comparing the protein profile obtained from first sample and the second sample to assess the change in the profile between the first selected time point and the second selected time point.

13. The method of claim 12, wherein the vessel disorder is a dissection.

14. The method of claim 14, wherein the dissection is an aortic dissection.

15. The method of claim 15, wherein the aortic dissection is Type A or Type B.

16. A method of diagnosing a subject suffering from an Type A aortic dissection comprising the steps of:
obtaining a blood sample from the subject;
determining in the sample the protein profile of claim 5;
comparing the protein profile of the same proteins in a healthy control, wherein an alteration in the levels of one or more proteins of the profile in the subject compared to the healthy control is indicative of a Type A aortic dissection.

17. A method of diagnosing a subject suffering from an Type B aortic dissection comprising the steps of:
obtaining a blood sample from the subject;
determining in the sample the protein profile of claim 6;
comparing the protein profile of the same proteins in a healthy control, wherein an alteration in the levels of a protein of the profile in the subject compared to the healthy control is indicative of a Type B aortic dissection.

18. A kit for detecting a vessel disorder comprising a container having the protein profile of claim 1.

19. A kit for detecting a vessel disorder comprising a container having the protein profile of claim 2.

20. A kit for detecting a vessel disorder comprising a container having the protein profile of claim 5.

21. A kit for detecting a vessel disorder comprising a container having the protein profile of claim 6.
